(19) United States
(12) Patent Application Publication
(10) Pub. No.: US 2003/0120332 A1
(43) Pub. Date: Jun. 26, 2003

(54) Stent graft with improved graft adhesion

(75) Inventor: David Ernest Hartley, Western Australia (AU)

Correspondence Address: COOK GROUP PATENT OFFICE, P.O. BOX 2269, BLOOMINGTON, IN, 47402

(73) Assignee: Cook Incorporated, Bloomington, IN (US)

(21) Appl. No.: 10/322,862

(22) Filed: Dec. 18, 2002

(30) Foreign Application Priority Data
Dec. 19, 2001 (AU) PR9617

(51) Int. Cl. A61F 2/06

(52) U.S. Cl. 623/1.13; 623/1.36; 623/1.52

(57) ABSTRACT

A graft (1) having a band of fibrous material (13) on an outer surface thereof adjacent its proximal end (9). The band of fibrous material extends circumferentially around the graft to promote adhesion. The band can be continuous around the proximal end of the graft. The band can be formed from a patch of fibrous material, cut or loop pile velour, by fibres woven or knitted into the material of the graft or by brushing of material of the graft to raise fibres of the material of the graft. The band of fibrous material has a length of from 5 mm to 15 mm along the length of the graft.

### CROSS-REFERENCE TO RELATED APPLICATIONS

This application claims priority of Australian provisional application Serial No. PR 9617, filed Dec. 19, 2001.

### TECHNICAL FIELD

This invention relates to endoluminal stent grafts and in particular to a stent graft for improving the adhesion or incorporation of such grafts into the wall of a lumen.

### BACKGROUND OF THE INVENTION

Throughout this specification when applied to a blood vessel the term distal, with respect to a stent graft or graft, is the end of the stent graft or graft furthest away in the direction of blood flow from the heart within a body lumen. The term proximal means the end of the stent graft or graft nearest to the heart in the direction of blood flow. Where the invention is applied to other lumens of the human or animal body then corresponding terms such as caudal and cranial should be understood.

A surgical stent graft or graft may be placed into a lumen in the body by endoluminal techniques or by surgical techniques. Such a stent graft or graft is constructed from biocompatible materials but it is desirable in the long term for such grafts to be actually incorporated into the wall of the body lumen and this requires that the tissue of the body lumen grows into the material of the graft.

It is known that tufts of fibrous material may be placed on grafts to enhance blood clotting and adhesion but such tufts do not promote complete circumferential adhesion. Particularly when such grafts are deployed endoluminally it is desirable that the amount of fibrous material be kept to a minimum to ensure that the graft can be compressed into small enough volume to enable deployment while at the same time ensuring good adhesion of the graft.

### SUMMARY OF THE INVENTION

The object of this invention is to provide a graft with material to ensure good adhesion or incorporation of the graft into a lumen.

In one form therefore the invention is said to reside in a graft or stent graft having a band of fibrous material on an outer surface thereof adjacent a distal or a proximal end thereof, the band of fibrous material extending circumferentially around the graft to promote adhesion and incorporation. Furthermore, the graft or stent graft can include separate bands of fibrous material extending circumferentially around the graft at both the distal and the proximal ends thereof to promote adhesion and incorporation.

Preferably the band is continuous around the proximal end of the graft.

In one embodiment the band is provided by patches of fibrous material around the proximal end of the graft. Alternatively the band of fibrous material is formed from velour or is provided by fibres woven or knitted into the material of the graft. The velour material may be loop pile material or cut pile material to present either loops of fibres or individual fibres to the wall of the vessel.

The band of fibrous material may alternatively be provided by brushing of the material of the graft to raise fibres of the material to provide the fibrous band.

In an alternative form, the invention may be said to reside in a graft having a continuous band of velour material adjacent a proximal end thereof, the continuous band of velour material extending circumferentially around the graft to promote adhesion.

In a further form, the invention may be said to reside in a continuous band of velour material around one end of a graft to provide adhesion against the wall of a lumen by ingrowth of the tissue of the wall into the velour material, the velour material being a continuous band around the graft.

In a further form, the invention may be said to reside in a stent graft adapted for insertion into an internal lumen of a patient to be incorporated therein, the stent graft comprising a substantially tubular body providing a fluid flow path and having a periphery defined by a biocompatible relatively impervious material wall, the wall having a proximal portion including a continuous external circumferential band of a fibrous biocompatible material.

In a further form, the invention may be said to reside in a stent graft adapted for implantation within a lumen in a human or animal body to reside therein, the stent graft having a proximal end and a distal end and a substantially tubular graft body formed from a relatively impervious biocompatible material, at least one stent to maintain the patency of the stent graft, and a continuous circumferential band of fibrous material on the outside of the tubular graft body adjacent the proximal end of the stent graft.

It would be seen that by the various forms of the invention there is provided a continuous band of velour material or fibrous material around the outside of the proximal end of a stent graft for good adhesion of that end of the stent graft to the wall of a lumen.

It will be noted that the graft of the present invention is particularly useful in delivery devices when endoluminal deployment is used because the band is of a relatively short length with respect to the length of the graft and hence does not make the device when it is compressed for deployment significantly larger over the entire length of the device.

The band of fibrous material can be a band which is stitched onto the graft at the proximal end, it may be fibres woven or knitted into the material of the graft or where the graft itself is manufactured from a fibrous woven, knitted or non-woven material at which the proximal area of the graft may be brushed to raise the fibres to provide the fibrous band.

To ensure good adhesion the band may be in the region of 5 mm to 15 mm in length along the length of the graft.

In one preferred form of the invention, the invention is particularly related to a stent graft using self-expanding stents such as stainless steel or nitinol stents.

The relatively impervious material may be a knitted or woven biocompatible material such as a dacron or similar material or it may be a non-woven fibrous material or it may be a plastics material sheet formed into a graft tube or extruded into tubular form.

It is desirable that the band be continuous around the proximal end of the graft so that with fibrous ingrowth of the cells of the wall of the lumen into the band of fibrous material on the graft, union of the wall and graft is obtained which provides sealing for blood flow which could occur between the graft and the wall as well as prevention of movement of the graft longitudinally within the lumen. In so doing there is improved incorporation of the graft into the wall of the lumen.

### BRIEF DESCRIPTION OF THE DRAWINGS

This then generally describes the invention but to assist with understanding reference will now be made to the accompanying drawing which show preferred embodiments of the invention. In the drawings,

FIG. 1 shows a perspective view of a stent graft including the adhesion or incorporation arrangement according to one embodiment of the present invention;

FIG. 2 shows a perspective view of a stent graft including the adhesion or incorporation arrangement according to an alternative embodiment of the present invention;

FIG. 3A shows a plan view of a stent graft shown in FIG. 1 with a first form of velour pile;

FIG. 3B shows a plan view of a stent graft shown in FIG. 1 with an alternative form of velour pile; and

FIG. 4 shows a detailed perspective view of a stent graft including an adhesion or incorporation arrangement of an alternative embodiment.

### DETAILED DESCRIPTION

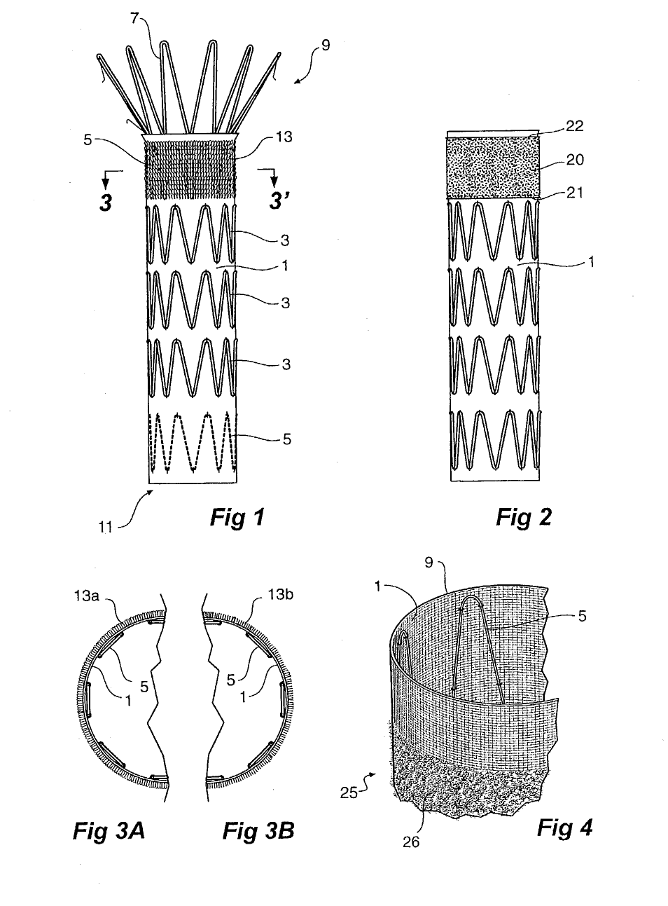

Looking more closely at FIG. 1 of the drawings it can be seen that in a stent graft comprising a graft material tube 1 which is held into a substantially cylindrical shape by means of external expandable stents 3 in the main body of the graft material 1 and internal stents 5 at each end of the graft. These stents can be self-expanding of, for example, commercially available Gianturco, zigzag or Z stent using a spring like metal such as stainless steel, nitinol, or other alloy metals. These stents can also be balloon or mechanically expandable stents, which are also commercially available.

The graft body has a proximal end 9 and a distal end 11.

In this particular embodiment, a proximally extending stent 7 is provided although this proximally extending stent is not essential to the invention.

Each stent 3, 5 and 7 is a Gianturco zig zag or Z stent manufactured from nitinol or stainless steel and is self expanding from a compressed state, in which the stent graft can be introduced into the lumen, to the expanded state shown.

Extending circumferentially around the graft body 1 at the proximal end 9 is a continuous circumferential region 13 of fibres extending from the graft material. The continuous circumferential region is formed on the material of the graft 1 by portions of the fibrous material woven into the material or extending from the woven graft material. The portions of fibrous material may be in the form of a loop pile or a cut pile to present either loops of fibres or individual fibres to the wall of the vessel when deployed within a body lumen.

The graft may have a diameter in the range of 25 to 50 millimetres and a length of from 100 to 200 millimetres. The band 13 may have a length of from 5 to 15 millimetres and may be adjacent the end of the stent graft or spaced from the proximal end of the graft by a distance of up to 10 millimetres.

FIG. 2 shows an alternative embodiment of the invention. In this embodiment the graft 1 is of substantially the same construction as shown in FIG. 1 except that it does not have a proximally extending uncovered stent. Around the proximal end 19 of the graft 1 is a continuous band of velour material 20 fastened to the graft 1 by stitching 22 at the proximal end and 21 at the distal end of the velour band 20. The velour provides loops of fibrous material which can be incorporated into the wall of the lumen into which the graft is placed by fibrous ingrowth. This provides adhesion of the graft into the lumen. One again the velour material may be of loop pile or cut pile form.

The provision of the continuous band at the proximal end of the graft provides adhesion and incorporation around the entire circumference of the graft. The adhesion and incorporation around the entire circumference of the graft will prevent blood or other body fluid, depending upon the lumen into which it is introduced, from getting behind the wall of the graft. The adhesion and incorporation will also assist with preventing movement of the graft within the lumen.

FIG. 3A and B show cross sectional plan views in the line 3-3′ on the stent graft shown in FIG. 1 but with different forms of velour pile.

FIG. 3A shows loop pile velour as the continuous band 13a of fibrous material around the circumference of the graft 1.

FIG. 3B shows cut pile velour as the continuous band 13b of fibrous material around the circumference of the graft 1.

FIG. 4 shows a detailed perspective view of part of a stent graft including an alternative adhesion arrangement for the proximal end of the stent graft. As with FIG. 1 the stent graft has a bio-compatible woven material body 1 with a self expanding stent 5 inside the graft body 1 at the proximal end 9 to provide a good contact of the graft material with the wall of a lumen into which it is deployed. In this case the continuous band of fibrous material 25 is provided by brushing or similar means to raise fibres 26 from the material of the graft 1. The brushing can be with a wire brush or a hooking system may be used to raise some of the fibres in a continuous band around the graft.

This brushing or hooking can also be used with non-woven biocompatible graft materials to raise a fibrous band around the circumference of a graft.

It may be noted that this specification has been particularly directed to the use of a velour band on the proximal end of a graft because that is the end of the graft which it is normally expected to get more blood pressure in the case where the lumen is a blood vessel and the proximal end in the case of an artery being the end nearer the heart. There may be situations, however, where the band of fibrous material may be placed around the distal end of a graft or at both ends and this invention is intended to cover those applications as well.

Throughout this specification various indications have been given as to the scope of this invention but the invention is not limited to any one of these but may reside in two or more of these combined together. The examples are given for illustration only and not for limitation.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

What is claimed is:

1. A stent graft comprising:
a tubular graft having a distal end, a proximal end, and an outer surface;
at least one expandable stent disposed on said tubular graft; and
a band of fibrous material on said outer surface and adjacent at least one of said distal end and said proximal end of said tubular graft, said band of fibrous material extending circumferentially around said graft.

2. The stent graft as in claim 1 wherein said band is continuous around said proximal end of said tubular graft.

3. The stent graft as in claim 1 wherein said band comprises a patch of fibrous material fastened around said proximal end of said tubular graft.

4. The stent graft as in claim 1 wherein said band of fibrous material includes velour material.

5. The stent graft as in claim 4 wherein said velour material includes a loop pile velour material.

6. The stent graft as in claim 4 wherein said velour material includes a cut pile velour material.

7. The stent graft as in claim 1 wherein said band of fibrous material includes fibres woven or knitted into material of said graft.

8. The stent graft as in claim 1 wherein said graft includes a graft material and wherein said band of fibrous material comprises brushed raised fibres of said graft material.

9. The stent graft as in claim 1 wherein said band of fibrous material has a length of from 5 mm to 15 mm along the length of said graft.

10. The stent graft as in claim 1 wherein said at least one expandable stent extends proximally from said proximal end of said tubular graft.

11. The stent graft as in claim 1 wherein said stent graft further comprises another band of fibrous material on said outer surface and adjacent a remaining one of said distal end and said proximal end of said tubular graft, said other band of fibrous material extending circumferentially around said graft.

12. A stent graft comprising:
a tubular graft having an outer surface and a proximal end;
at least one expandable stent disposed on said tubular graft; and
a continuous band of velour material adjacent said proximal end of said tubular graft, said continuous band of velour material extending circumferentially around said graft.

13. The stent graft as in claim 12 wherein said continuous band of velour material is disposed on said outer surface of said tubular graft.

14. A stent graft adapted for insertion into an internal lumen of a patient to be incorporated therein, the stent graft comprising a substantially tubular body providing a fluid flow path and having a periphery defined by a biocompatible relatively impervious material wall, the wall having a proximal portion including a continuous external circumferential band of a fibrous biocompatible material.

15. The stent graft as in claim 14 wherein the stent graft further comprises at least one stent to maintain the patency of the stent graft, and a continuous circumferential band of fibrous material on the outside of the tubular body adjacent the proximal end of the stent graft.
